(19) United States
(12) Patent Application Publication
(10) Pub. No.: US 2006/0167424 A1
(43) Pub. Date: Jul. 27, 2006

(54) Absorbent article having pulpless absorbent core

(76) Inventors: Edward Kuo-Shu Chang, Charlotte, NC (US); Patrick King Yu Tsang, Matlock (GB); Andrew Wright, Pilsey (GB)

Correspondence Address: FULBRIGHT & JAWORSKI L.L.P., 80 SOUTH EIGHTH STREET, SUITE 2100, MINNEAPOLIS, MN, 55402, US

(21) Appl. No.: 11/322,834

(22) Filed: Dec. 30, 2005

Related U.S. Application Data
(60) Provisional application No. 60/640,918, filed on Dec. 31, 2004.

(51) Int. Cl. A61F 13/15

(52) U.S. Cl. 604/368; 604/378

(57) ABSTRACT

A disposable absorbent article comprising a liquid permeable topsheet, a liquid impermeable backsheet, and an absorbent core interposed between the topsheet and the backsheet. The absorbent member is defined by a pulp-less absorbent core having a superabsorbent material providing efficient fluid handling characteristics. The absorbent core may be defined by its fluid handling properties including, but not limited to, greater absorbency rate index (ARI) and greater percentage absorbency rate index (PARI) as compared to absorbent articles of conventional pulp-containing technology.

### RELATED APPLICATION

This application claims the benefit of U.S. Provisional Application No. 60/640,918, filed Dec. 31, 2004, the entire contents being incorporated herein by reference.

### FIELD OF THE INVENTION

The invention relates to disposable pulpless absorbent articles including superabsorbent materials which absorb water and aqueous liquids and provide improved properties, in particular faster absorption time, while maintaining acceptable fluid capture and retention properties.

### BACKGROUND OF THE INVENTION

For fit, comfort and aesthetic reasons and from environmental aspects, there is an increasing trend to make disposable absorbent articles smaller and thinner. Disposable absorbent articles often contain superabsorbent materials and fiber or fluff to improve liquid handling characteristics of the articles. One may propose that the physical size of such articles could simply be reduced by decreasing the content of the high volume fluff fiber within the articles. However, since fiber acts to quickly, but temporarily, absorb liquid insults prior to capture by the superabsorbent material, a reduction of fiber content may lead to unacceptable fluid handling characteristics, such as liquid leakage, slow liquid capture and gel blocking.

Superabsorbent refers to a water-swellable, water-insoluble, organic or inorganic material capable of absorbing at least about 10 times its weight and up to about 30 times its weight in an aqueous solution containing 0.9 weight percent sodium chloride solution in water. A superabsorbent polymer is a crosslinked neutralized polymer which is capable of absorbing large amounts of aqueous liquids and body fluids, such as urine or blood, with swelling and the formation of hydrogels, and of retaining them under a certain pressure in accordance with the general definition of superabsorbent. The superabsorbent polymers that are currently commercially available are crosslinked polyacrylic acids or crosslinked starch-acrylic acid graft polymers, in which some of the carboxyl groups are neutralized with sodium hydroxide solution or potassium hydroxide solution. As a result of these characteristic properties, these polymers are chiefly used for incorporation into sanitary articles, such as babies' diapers, incontinence products and sanitary towels.

In future absorbent article constructions, it is expected that there will be less fiber material, or potentially none at all. The superabsorbent polymer of future diaper constructions must have a sufficiently high absorption rate to maximize the use of available capillary spaces and to compensate for the reduction or substantial elimination of typically fast absorbing fibers.

### SUMMARY OF THE INVENTION

The present invention is directed to disposable pulpless absorbent articles including superabsorbent materials which absorb water and aqueous liquids and provide improved properties, in particular faster absorption time while maintaining acceptable fluid retention properties. To offset a reduction in fiber levels an improved superabsorbent material may be necessary in order to provide acceptable fluid handling characteristics, such as equivalent total retention capacity of body fluids, etc.

Disposable absorbent articles according to the present invention have higher centrifugal retention capacity (CRC) values, higher absorbency rate index (ARI) values and/or higher percentage absorbency rate index (PARI) as compared to products manufactured with existing pulp-containing technologies.

It is therefore an object of the present invention to provide a disposable absorbent article having a superabsorbent composition with an increased rate of liquid absorption, particularly within the targeted insult region. It is another object is to maintain acceptable liquid handling properties and liquid retention characteristics even when the fiber content is reduced or eliminated and the amount of superabsorbent material is increased in percent by weight based on the absorbent structure. This may be achieved by increasing the absorption rate of the superabsorbent polymer. Additionally, the selective placement of superabsorbent material within the absorbent core may yield benefits.

Absorbent articles according to the present invention contain minimal or no fluff material in order to address the increasing trend to make sanitary articles smaller and thinner. To offset a reduction in fiber levels an improved superabsorbent material may be used to provide acceptable fluid handling characteristics, such as equivalent total retention capacity of body fluids.

The present invention is also directed to a method of manufacturing a pulpless disposable absorbent article having acceptable or better fluid handling capabilities as compared disposable absorbent articles containing fiber or fluff. These and other objects of the invention will be more readily apparent when considered in reference to the following description and when taken in conjunction with the accompanying drawings.

Still other embodiments of the inventive disposable article and their manufacturing methods will become readily apparent to those skilled in the relevant art from the following detailed description of the drawings, wherein the various embodiments of the invention are described by way of illustrating the best mode contemplated for carrying out the invention. The invention is capable of other and different embodiments, its several details are capable of modification and its several structural or processed details are capable of modification in various and obvious respects, all without departing from the spirit and the scope of the present invention. Accordingly, the following drawings and detailed description of the drawings are to be regarded as illustrative in nature and not restrictive.

### BRIEF DESCRIPTION OF THE DRAWINGS

The present invention will be more fully understood and further advantages will become apparent when reference is made to the following detailed description of the invention and the accompanying drawings wherein like numerals represent like elements, and in which:

FIG. 1 representatively shows a perspective view of an a disposable absorbent article of the present invention.

FIG. 2 depicts a top plan view of disposable absorbent article.

FIG. 3 depicts a top plan view of the disposable absorbent article of FIG. 2.

FIG. 4 depicts top plan views of forms of disposable absorbent articles suitable for application of the present invention.

FIG. 5 is a graphical representation of the CRC values over time for the various samples of Table 1.

FIG. 6 is a graphical representation of the CRC values in percentage form for the various samples of Table 1.

### DETAILED DESCRIPTION

The present invention concerns a disposable absorbent article having an absorbent core structure used to absorb and retain liquids, such as urine or blood. The absorbent core structure is typically, or forms typically part of, a disposable absorbent article, such as preferably diaper and training pants, sanitary napkins, panty liners, and adult incontinence products.

The absorbent core structure forms part of a disposable absorbent article which is adapted to be worn adjacent to the body of a wearer to absorb and contain various exudates discharged from the body. The absorbent article may be configured to closely conform to the body of the wearer to assist in effective containment of body exudates.

In one embodiment, an absorbent article of the present invention will be described in terms of a disposable diaper which is adapted to be worn about the lower torso of a child. It is understood that the articles and methods of the present invention are equally adaptable for other types of absorbent articles, such as adult incontinent products, training pants, feminine hygiene products, other personal care or health care garments, and the like.

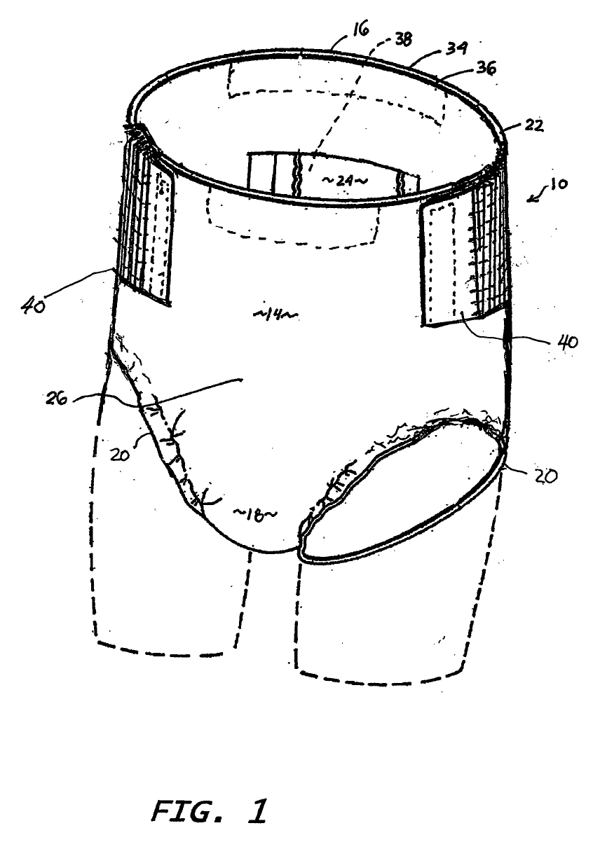

FIG. 1 representatively shows one example of a disposable absorbent article generally indicated at 10 and incorporating aspects of the present invention. FIG. 1 illustrates a diaper in a perspective view as worn by a user.

The illustrated diaper 10 includes a body 12 which generally defines a front waist region 14, a rear waist region 16, and a crotch region 18 which extends between and connects the front and rear waist regions 14 and 16. The body 12 further defines a pair of laterally opposed side edges 20, a pair of longitudinally opposed waist edges 22, an interior body facing surface 24 which is configured to contact the wearer, an outer garment facing surface 26 opposite the interior surface 24 which is configured to contact the wearer's clothing in use. The outer surface 26 may be defined by an outer cover layer 34 and the interior surface 24 may be defined by a body side liner 36. Diaper 10 includes an absorbent core 38 which may be located between the outer cover 34 and the body side liner 36.

The front waist region 14 comprises the portion of the diaper 10 which, when worn, is positioned on the front of the wearer while the rear waist region 16 comprises the portion of the diaper 10 which, when worn, is positioned on the back of the wearer. The crotch region 18 comprises the portion of the diaper 10 which, when worn, is positioned between the legs of the wearer and covers the lower torso of the wearer. The laterally opposed side edges 20 of the diaper 10 generally define portions of the leg openings. The waist edges 22 of the absorbent body 12 of the diaper 10 are configured to encircle the waist of the wearer when worn and provide a waist opening when fastened that defines a waist perimeter dimension.

The disposable absorbent article according to the present invention provides a close fitting seal around the thighs of the user, thereby significantly improving its leakage prevention capability. The close fitting seal of the inventive garment is at least partially provided through addition of fastening element 40. The fastening element 40 may include known adhesive elements or hook fastening devices. A hook fastening element may be selected so as to be engageable with the loops formed on the surface of a nonwoven fabric. Thus, the nonwoven material of the stretchband panel provides the loop element of a hook and loop fastening system similar to those generally known in the art. In further embodiments, a loop landing tape may be located near the front waist region of the outside face of the inventive article, and a pair of hook fastening elements may be located near the rear waist region. The loop landing tape may be constructed from a knitted, extruded, or non-woven material, as is generally known in the art. A variety of fastener systems and devices are known to those of ordinary skill in the art. A particular fastener choice would not otherwise alter the scope of the appended claims.

The diaper 10 may further include leg elastics, containment flaps, and waist elastics as are known to those skilled in the art. For example, the absorbent body 12 of the disposable diaper 10 may include a pair of containment flaps which are configured to provide a barrier to the lateral flow of exudates. The containment flaps may be located along the laterally opposed side edges 20 of the absorbent body 12.

The disposable diaper 10 may further optionally include elastics at the waist edge 22 of the diaper 10 to further prevent leakage of body exudates. For example, the disposable diaper may further comprise elastic waist features that help provide improved fit and containment of body exudates.

A diaper 10 according to the present invention may also include a pair of leg elastic members which are connected to the laterally opposed side edges 20 in the crotch region 18 of the diaper 10. The leg elastics are generally adapted to fit about the legs of a wearer in use to maintain a positive, contacting relationship with the wearer to effectively reduce or eliminate the leakage of body exudates from the diaper 10. Materials suitable for use as the leg elastics and waist elastic are well known to those skilled in the art. Exemplary of such materials are sheets or strands or ribbons of a polymeric, elastomeric material which are adhered to the outer cover 34 in a stretched position, or which are attached to the outer cover 34 while the outer cover is pleated, such that elastic constrictive forces are imparted to the outer cover 34. The leg elastics may also include such materials as polyurethane, synthetic and natural rubber. Leg elastics, containment flaps and waist elastics may include elastic foam materials, elastic films (apertured, woven and non-woven, for example), elastic scrim material, elastic non-woven materials, elastic composites, and selectively activated elastic materials.

The diaper 10 may be of various suitable shapes. For example, in the unfastened configuration, the diaper may have an overall rectangular shape, T-shape or an approximately hourglass shape. The various aspects and configurations of the invention can provide distinctive combinations of softness, body conformity, reduced red-marking of the wearer's skin, reduced skin hydration, improved containment of body exudates and improved aesthetics.

The various components of the diaper 10 may be integrally assembled together employing various types of suitable attachment means, such as adhesive, sonic and thermal bonds or combinations thereof. In the illustrated shown embodiments, for example, the outer cover 34 and body side liner 36 are joined to each other. Similarly, other diaper components, such as the leg elastics and primary fasteners, may be assembled into the diaper 10 by employing the above-identified attachment mechanisms. Desirably, the majority of the diaper components are assembled together using ultrasonic bonding techniques for reduced manufacturing cost and improved performance.

The outer cover 34 of the diaper 10 may suitably be composed of a material which is either liquid permeable or liquid impermeable. It is generally preferred that the outer cover 34 be formed from a material that is substantially impermeable to liquids. A typical outer cover can be manufactured from a thin plastic film or other flexible liquid-impermeable material. For example, the outer cover 34 may be formed from a polyethylene film. If it is desired to present the outer cover 34 with a more cloth-like feeling, the outer cover 34 may comprise a polyolefin film having a nonwoven web laminated to the outer surface thereof, such as a spunbond web of polyolefin fibers. For example, a stretch-thinned polypropylene film may have thermally laminated thereto a spunbond web of polypropylene fibers. Methods of forming such cloth-like outer covers are known to those skilled in the art.

Further, the outer cover 34 may be formed of a woven or nonwoven fibrous web layer which has been totally or partially constructed or treated to impart a desired level of liquid impermeability to selected regions that are adjacent or proximate the absorbent core 38. Still further, the outer cover 34 may optionally be composed of a micro-porous “breathable” material which permits vapors to escape from the absorbent core 38 while still preventing liquid exudates from passing through the outer cover 34. For example, the outer cover 34 may comprise a stretched microporous polyolefin film having a nonwoven web laminated to the outer surface thereof, such as a spunbond web of polyolefin fibers. The outer cover 34 can also be embossed or otherwise provided with a matte finish to provide a more aesthetically pleasing appearance.

The bodyside liner 36 suitably presents a bodyfacing surface which is compliant, soft feeling, and nonirritating to the wearer's skin. Further, the bodyside liner 36 may be less hydrophilic than the absorbent core 38, to present a relatively dry surface to the wearer, and may be sufficiently porous to be liquid permeable, permitting liquid to readily penetrate through its thickness. A suitable bodyside liner 36 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The bodyside liner 36 is suitably employed to help isolate the wearer's skin from liquids held in the absorbent core 38.

Various woven and nonwoven fabrics can be used for the bodyside liner 36. For example, the bodyside liner maybe composed of a meltblown or spunbonded web of polyolefin fibers. The bodyside liner may also be a bonded-carded web composed of natural and/or synthetic fibers. The bodyside liner may be composed of a substantially hydrophobic material, and the hydrophobic material may, optionally, be treated with a surfactant or otherwise processed to impart a desired level of wettability and hydrophilicity. The bodyside liner 36 may further include a lotion or treatment applied thereto to which is configured to treat or be transferred to the wearer's skin.

The absorbent core 38 of the diaper 10 contains particles of a high-absorbency material commonly known as superabsorbent material. In a particular embodiment, the absorbent core 38 comprises superabsorbent hydrogel-forming particles. The superabsorbent particles may be selectively placed into desired zones of the absorbent core 38 to better contain and absorb body exudates. The concentration of the superabsorbent particles may also vary through the thickness of the absorbent core 38. Alternatively, the absorbent core 38 may comprise a laminate of fibrous webs and superabsorbent material or other suitable means of maintaining a superabsorbent material in a localized area.

The high-absorbency material can be selected from natural, synthetic, and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble but swellable. Such means can include, for example, physical entanglement, crystalline domains, covalent bonds, ionic complexes and associations, hydrophilic associations such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthum gum, locust bean gum, and the like. Mixtures of natural and wholly or partially synthetic absorbent polymers can also be useful in the present invention. Such high-absorbency materials are well known to those skilled in the art and are widely commercially available.

The high absorbency material may be in any of a wide variety of geometric forms. As a general rule, it is preferred that the high absorbency material be in the form of discrete particles. However, the high absorbency material may also be in the form of fibers, flakes, rods, spheres, needles, or the like. As a general rule, the high absorbency material is present in the absorbent body in an amount of from about 85 to about 100 weight percent based on total weight of the absorbent core 38.

One superabsorbent material suitable for use in an embodiment of the present invention has a vortex time of approximately 30 seconds. Alternative superabsorbent materials having similar or higher vortex times may also be practicable. A range of vortex times for superabsorbent materials which may used to practice the present invention is between 10 and 120 seconds.

The superabsorbent material may be selectively placed within the absorbent core 38 so that more material is within the target zone or portions thereof as compared to portions of the core 38 outside of the target zone. Additionally, the amount of superabsorbent material or even the composition of a superabsorbent material may vary depending on the particular zone or portion of the core 38.

The absorbent core 38 may have any of a number of shapes. For example, the absorbent core may be rectangular, I-shaped, or T-shaped. In the embodiment of FIG. 1, the absorbent core is generally rectangular in shape. The size and the absorbent capacity of the absorbent core 38 should be compatible with the size of the intended wearer and the liquid loading imparted by the intended use of the absorbent article.

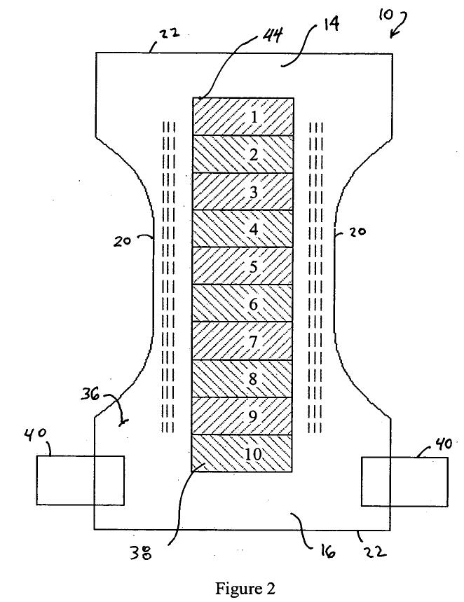

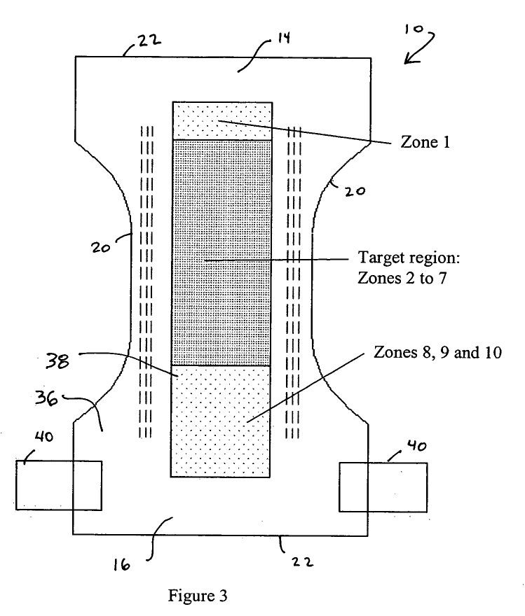

FIG. 2 illustrates a disposable absorbent article 10 including an absorbent core 38 divided into ten generally equally spaced sections or zones numbered one through ten. FIG. 3 discloses the target region of the article 10 defined between approximately Zone 2 through Zone 7. In this example, the target region of the absorbent article is generally defined as the portion between the 10% to 80% dimensions as measured from the front edge 44 of the absorbent core 38. The absorbent article 10 of FIG. 3 is a general representation of an absorbent article and alternative sizes, shapes and features would be appreciated by those of ordinary skill in the art. For example, the side edges of an alternative embodiment of an absorbent article according to the present invention may be concave or otherwise non-linearly configured.

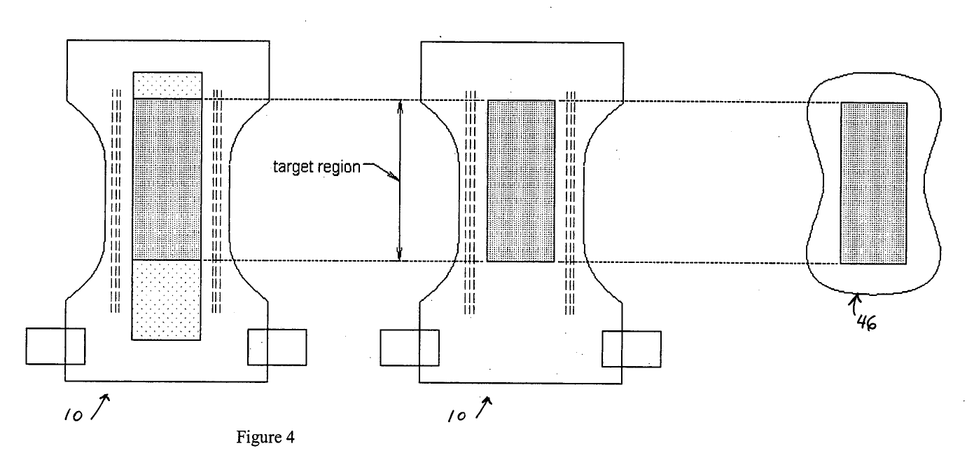

FIG. 4 illustrates various alternative embodiments of absorbent articles 10 and depicts target zones for these embodiments. As illustrated, the target regions of the products may be defined as a portion of the overall absorbent core 38 or even as the entire absorbent core 38 (as shown in adult incontinence pad 46). The insult target region is the primary region receiving liquid or fecal insults during product use. The target insult region may be defined, as shown in FIGS. 2 and 3, to include multiple zones of the absorbent core 38.

Table 1 includes Centrifugal Retention Capacity (CRC) data of various current absorbent products and a preferred embodiment of the present invention, referred to as “Working Product T3CM”. Less preferred embodiments utilizing aspects of the present invention are referred to as Experimental Products A and B. Referring again to FIG. 2, the CRC data was compiled for various size 4 (10 kg-17 kg) diapers divided into ten equal length zones. The CRC was measured at 0.5, 1, 2, 4 and 30 minutes.

The CRC data of Table 1 was collected by experimentation according to the following test protocol.

Zoned Centrifuge Retention Capacity Test

Equipment and materials:

1. Large plastic tray

2. Hanging apparatus with clips

3. Plastic weighing pans

4. Measuring rule

5. Fine tip, permanent marker pen

6. Scissors

7. Timer

8. 0.9% saline solution

9. Electronic top loading scale accurate to 0.01 gm.

10. Centrifuge (1400 rpm) with drain

Procedure
1. Position the plastic tray under the hanging apparatus. Fill with an excess amount of 0.9% saline solution.
2. Weigh 14 diapers and record the weight of each diaper as WI.
3. Measure the length of the core of each diaper and divide into 10 sections of equal length. Using a permanent, fine tip marker pen, draw a line marking the longitudinal edge of each of the 10 sections.
4. Starting from the front of the diaper number the sections 1 through to 10, with 10 being the rearmost section.
5. Take two of the weighed and marked diapers and immerse in the saline solution for 30 seconds. Ensure that during this time the diapers are fully immersed.
6. After the allotted time, remove the diapers from the saline solution and immediately transfer to the centrifuge, placing the diapers in the centrifuge with the nonwoven against the centrifuge wall. Start the centrifuge and spin for 3 minutes.
7. Remove the diapers from the centrifuge, place the diapers in the tared weighing pans and record each weight as WF.
8. With the scissors carefully cut along the lines separating each of the ten sections of the diaper. Weigh each divided section of the diaper, gathering up any pieces of core material that may have dropped out of each section during cutting and including in these in the weight measurement. Record the weight of each section as Sn, where n is the section number.
9. Repeat steps 5 to 8 but successively increase the immersion time to 1 minute, 2 minutes, 4 minutes, 10 minutes and 30 minutes.
10. Finally, take two weighed, diapers and divide into the marked sections. Weigh each section to record the dry, unswollen weight of each section as Dn.
11. Perform calculations:
   Overall centrifuge retention capacity CRC=WF−WI
   Retention capacity per section at time t=Sn−Dn

TABLE 1
Core profile and CRF absorbency rate per zone

| | Immersion | CRC | FRONT [symbol] Centrifuge Retention Capacity per section (g) → REAR | | | | | | | | | |
|---|---|---|---|---|---|---|---|---|---|---|---|---|
| Product | Time (min) | (g) | 1 | 2 | 3 | 4 | 5 | 6 | 7 | 8 | 9 | 10 |
| Experimental | 0.5 | 67 | 4.7 | 7.8 | 7.6 | 8.2 | 7.8 | 7.6 | 7.6 | 4.5 | 4.5 | 3.6 |
| product A | 1 | 82 | 6.5 | 9.7 | 9.4 | 9.3 | 9.7 | 9.8 | 8.6 | 5.6 | 5.4 | 4.1 |
| | 2 | 109 | 8.2 | 11.4 | 12.8 | 14.0 | 12.7 | 13.2 | 11.8 | 8.5 | 7.2 | 5.5 |
| | 4 | 216 | 18.1 | 22.6 | 23.3 | 24.4 | 26.0 | 24.0 | 22.6 | 19.8 | 18.7 | 13.0 |
| | 30 | 538 | 55.6 | 70.2 | 72.7 | 70.0 | 62.3 | 56.6 | 50.8 | 32.1 | 42.3 | 23.0 |
| Pampers | 0.5 | 83 | 9.6 | 9.1 | 9.5 | 10.8 | 11.3 | 7.7 | 5.7 | 5.8 | 5.7 | 6 |
| Baby Dry | 1 | 116 | 12.9 | 15.7 | 14.4 | 16.2 | 12.6 | 12.7 | 7.7 | 7.4 | 8.5 | 8 |
| (10-17 kg) | 2 | 111 | 12.3 | 12.9 | 12.9 | 14.3 | 14.3 | 12.4 | 6.7 | 7.1 | 8.2 | 8.1 |
| | 4 | 152 | 15.5 | 20.8 | 20.6 | 15.8 | 21.9 | 18 | 11.3 | 9.5 | 8.9 | 8.7 |
| | 30 | 281 | 33.2 | 36.6 | 37.9 | 36 | 33.7 | 29.5 | 22.5 | 12.4 | 16.2 | 21.2 |
| Luvs - Ultra | 0.5 | 91 | 7.8 | 10.7 | 8.2 | 10.7 | 11.2 | 11.4 | 9.3 | 7.2 | 7.3 | 6 |
| Leakguard | 1 | 103 | 9.2 | 10.5 | 12.8 | 12.9 | 10.6 | 11 | 9.5 | 8.8 | 7.7 | 7.8 |
| (10-17 kg) | 2 | 125 | 8.5 | 13.6 | 14.3 | 15.4 | 13.2 | 15.4 | 14 | 10.3 | 9.1 | 9.8 |
| | 4 | 158 | 15.3 | 16.4 | 17.7 | 24.5 | 16.4 | 21.3 | 15 | 9.4 | 11.3 | 8.6 |
| | 30 | 265 | 20.4 | 23.8 | 28.1 | 31.1 | 26.9 | 24.1 | 29.4 | 20.4 | 27.7 | 30.3 |
| Huggies | 0.5 | 125 | 11.1 | 13.2 | 14.9 | 12.8 | 12.3 | 15.9 | 12.8 | 11.3 | 10.3 | 9.4 |
| Baby | 1 | 146 | 11.1 | 16.8 | 15.5 | 14.3 | 15.5 | 16.5 | 17.2 | 13.4 | 13.3 | 10.7 |
| Shaped | 2 | 187 | 16 | 21.4 | 20.6 | 18.8 | 18.4 | 20.6 | 21.2 | 16.4 | 17.8 | 14 |
| (10-17 kg) | 4 | 234 | 18.6 | 28 | 30.7 | 29.2 | 22.1 | 28 | 22.5 | 18.3 | 19.2 | 16.8 |
| | 30 | 266 | 20.5 | 32.9 | 33.5 | 33.4 | 25 | 29.8 | 28.4 | 24 | 19.9 | 16.7 |
| White | 0.5 | 127 | 12.1 | 16 | 14.4 | 14.1 | 12.2 | 14.8 | 13.4 | 10.2 | 9.3 | 9.5 |
| Cloud | 1 | 160 | 14.7 | 17.2 | 20.1 | 20.5 | 19.2 | 17.3 | 13.8 | 11.1 | 11.8 | 12.8 |
| Diaper (10-17 kg) | 2 | 188 | 17.6 | 16.9 | 19.2 | 20.6 | 22.7 | 19 | 18.4 | 15.1 | 17.7 | 18.9 |
| | 4 | 208 | 22.3 | 21.6 | 24.8 | 23.1 | 20.3 | 20.5 | 20.9 | 15.8 | 16.7 | 19.6 |
| | 30 | 330 | 35.3 | 35.8 | 39.9 | 35.7 | 36.5 | 36.5 | 27.4 | 26.4 | 24.9 | 27.2 |
| Experimental | 0.5 | 191 | 19.1 | 19.1 | 19.1 | 19.1 | 19.1 | 19.1 | 19.1 | 19.1 | 19.1 | 19.1 |
| product B | 1 | 251 | 25.1 | 25.1 | 25.1 | 25.1 | 25.1 | 25.1 | 25.1 | 25.1 | 25.1 | 25.1 |
| | 2 | 294 | 29.4 | 29.4 | 29.4 | 29.4 | 29.4 | 29.4 | 29.4 | 29.4 | 29.4 | 29.4 |
| | 4 | 350 | 35.0 | 35.0 | 35.0 | 35.0 | 35.0 | 35.0 | 35.0 | 35.0 | 35.0 | 35.0 |
| | 30 | 420 | 42.0 | 42.0 | 42.0 | 42.0 | 42.0 | 42.0 | 42.0 | 42.0 | 42.0 | 42.0 |
| Working | 0.5 | 190 | 11.7 | 22.9 | 24.2 | 24.3 | 24.3 | 25.6 | 22.6 | 13.3 | 12.4 | 6.9 |
| product | 1 | 232 | 14.3 | 28.4 | 28.9 | 30.7 | 29.3 | 30.6 | 28.2 | 15.4 | 15.5 | 9.0 |
| T3CM | 2 | 308 | 17.1 | 35.7 | 39.9 | 39.1 | 40.2 | 40.1 | 36.5 | 22.4 | 20.2 | 18.1 |
| | 4 | 374 | 23.9 | 40.8 | 48.8 | 48.5 | 48.2 | 47.4 | 43.1 | 24.8 | 32.4 | 14.5 |
| | 30 | 483 | 46.4 | 52.7 | 59.9 | 62.7 | 54.9 | 57.3 | 54.0 | 39.4 | 43.4 | 14.4 |

Table 1 illustrates that Working Product T3CM had substantially greater CRC values for each zone as compared to currently available diapers. For example, the CRC value for zone 5 of the Working Product T3CM was 54.9 grams at 30 minutes while the CRC values of zone 5 of the Pampers Baby Dry, Luvs—Ultra Leakguard and Huggies Baby Shaped products were 33.7 grams, 26.9 grams and 25 grams, respectively.

It was determined through consumer use testing that a pulpless product (Experimental Products A & B) that has similar CRC values as current fluff/SAP products would have an insufficient lock up rate. The performance of the Working Product T3CM is achieved through selective zoning and use of a rapid vortex rate SAP.

One skilled in the art would recognize that a superabsorbent material with a fast vortex rate (less than 40 sec.) used in a conventional fluff/SAP core will lead to gel blocking and premature product failure. Whereas in the Working Product T3CM a fast vortex rate SAP yields acceptable performance without gel blocking.

Each of currently available products of Table 1 includes conventional pulp fibers acts (which act as temporary reservoirs to capture liquid prior to absorption by the superabsorbent material). In comparison, products according to the present invention are pulpless.

In another aspect of the invention, the absorbent core by weight contains between 0% to 15% pulp, 55% to 100% superabsorbent, 0% to 45% adhesive or binder or other additive, 0% to 45% nonwoven. A more preferred range of pulp is between 0% and 10% and a yet more preferred range of pulp is between 0% and 5%. As used herein, the term “pulpless absorbent core” is defined to mean an absorbent core having less than approximately 15% pulp. Pulp can include hydrophilic fibers such as: cellulose fibers, for example, mechanical pulp, chemical pulp, semichemical pulp, digested pulp, as obtained from wood; and artificial cellulose fibers, for example, rayon, acetates. Among the above-exemplified fibers, cellulose fibers are widely preferred in existing products. In addition, the hydrophilic fibers may comprise synthetic fibers such as polyamides, polyesters, and polyolefins. Pulp is not limited to the above-exemplified hydrophilic fibers.

Table 2 provides Absorbency Rate Index values for the tested product samples of Table 1. The Absorbency Rate Index (ARI) is defined as:

ARI (zone 2 to 7)=Sum of CRC (zone 2 to 7 at 0.5, 1, 2 and 4 minutes)

For example, the ARI of the Pampers Baby Dry product is the sum of: 54, 79, 74 and 108, or 315. In comparison the ARI of the Working Product T3CM is 828 (144+176+231+277). Table 2 provides minimum ARI values for a Target Desired Rate (720), a more preferred desired rate (756), and a yet more preferred desired rate (815).

TABLE 2
Core absorbency rate in Zone 2 to 7 (Sum of Zone 2 to 7 CRC)

| | | | | | | | | | | Even |
|---|---|---|---|---|---|---|---|---|---|---|
| | | Pampers | Luvs - Ultra | Huggies | | | | | More | more |
| | Ex- | Baby Dry | Leakguard | Baby | White Cloud | | Working | Target | desired | desired |
| | perimental | (10-17 kg) | (10-17 kg) | Shaped | Diaper | Experimental | product | desired | rate or | rate or |
| | product A | (US) | (US) | (10-17 kg) | (10-17 kg) | product B | T3CM | rate | higher | higher |
| Immersion | | | | | | | | | | |
| time (min) | | | | | | | | | | |
| 0.5 | 47 | 54 | 62 | 82 | 85 | 115 | 144 | 135 | 138.6 | 150 |
| 1 | 57 | 79 | 67 | 96 | 108 | 151 | 176 | 150 | 159.6 | 180 |
| 2 | 76 | 74 | 86 | 121 | 117 | 176 | 231 | 195 | 205.8 | 225 |
| 4 | 143 | 108 | 111 | 161 | 131 | 210 | 277 | 240 | 252.0 | 260 |
| 30 | 383 | 196 | 163 | 183 | 212 | 252 | 341 | 320 | 325.0 | 330 |
| Absorbency | | | | | | | | | | |
| Rate Index | | | | | | | | | | |
| 0 to 4 min | 322 | 315 | 326 | 459 | 441 | 652 | 828 | 720 | 756 | 815 |

Table 3 provides comparison between a significantly larger diaper (15-18 kg) and the minimum ARI values for a Target Desired Rate (720), a more preferred desired rate (756), and a yet more preferred desired rate (815). Table 3 illustrates that a pulpless absorbent article according the present invention has an ARI value that is greater than the ARI of a substantially larger product, i.e., White Cloud Training Pant (15-18 kg).

TABLE 3
Core absorbency rate in Zone 2 to 7 (Sum of Zone 2 to 7 CRC)

| | White Cloud | Target | | Even more |
|---|---|---|---|---|
| | Training Pants | desired | More desired | desired |
| | (15-18 kg) | rate | rate or higher | rate or higher |
| Immersion | | | | |
| time (min) | | | | |
| 0.5 | 139 | 135 | 138.6 | 150 |
| 1 | 150 | 150 | 159.6 | 180 |
| 2 | 196 | 195 | 205.8 | 225 |
| 4 | 205 | 240 | 252.0 | 260 |
| 30 | 293 | 320 | 325.0 | 330 |
| Absorbency | | | | |
| Rate Index | | | | |
| 0 to 4 min | 690 | 720 | 756 | 815 |

One important aspect of the present invention is the realization that the ARI of a pulpless product must be significantly greater than ARI values of prior absorbent articles which incorporated pulp within an absorbent core. For the conventional fluff/SAP core, additional, though temporary, fluid capacity is provided by the fluff. Whereas in a pulpless core, fluid capacity is achieved solely through the SAP material and not through the fluid capacity of the fluff, i.e., there is little or no temporary fluid storage for a pulpless core. Since a pulpless absorbent core may not effectively wick liquid, the insult target region absorbency rate of an article according to the present invention must be sufficient high in order to prevent premature leakage.

Zones 2 to 7 effectively define the target insult region of the typical absorbent article in diaper form. Since a pulpless absorbent does not effectively wick fluid, the absorbent product's target region absorbency rate must be sufficient high to prevent premature leakage. Table 2 shows that current conventional diapers all have substantially lower target zone CRC values than a product according to the present invention, e.g., Working Product T3CM. In a preferred embodiment, a pulpless absorbent article according to the present invention provides an ARI value of 700 grains or greater in order to prevent premature leakage.

Table 4 provides core absorbency rate data expressed in percentage form. For each sample, a percentage of absorbency at time intervals of 0.5, 1, 2 and 4 minutes was obtained by dividing the amount absorbed at a given time interval by the total amount absorbed at 30 minutes. For example, the core absorbency rate data expressed in percentage form for the Working Product T3CM at 0.5 minutes was calculated by dividing 144 (from Table 2) by 483 (from Table 1), or 30%. Continuing with this example, the core absorbency rate data expressed in percentage form at 4 minutes was calculated by dividing 277 (Table 2) by 483 (Table 1), or 57%.

Table 4 also contains the Percentage Absorbency Rate Index (PARI) from 0 to 4 minutes for the various samples where PARI is defined as the following;

PARI (zone 2 to 7)=Sum of percentage CRC (zone 2 to 7) at 0.5, 1, 2 and 4 minutes. For example, the PARI value of the Working Product T3CM is calculated as follows: PARI=30+36+48+57=171%.

Table 4 also discloses targeted preferred ARI and PARI values for embodiments of the present invention. As suggested by Table 4, a pulpless absorbent product according to the present invention would preferably have a PARI value of approximately 170% or greater in order to prevent premature leakage.

TABLE 4
Core absorbency rate in Zone 2 to 7 (Sum of Zone 2 to 7 CRC) expressed as a percentage of total
absorbency

| | | | | | | | | | | Even |
|---|---|---|---|---|---|---|---|---|---|---|
| | | | Luvs - | Huggies | White | | | | More | more |
| | Ex- | Pampers | Ultra | Baby | Cloud | | Working | Target | desired | desired |
| | perimental | Baby Dry | Leakguard | Shaped | Diaper | Experimental | product | desired | rate or | rate or |
| | product A | (10-17 kg) | (10-17 kg) | (10-17 kg) | (10-17 kg) | product B | T3CM | rate | higher | higher |
| Immersion | | | | | | | | | | |
| time (min) | | | | | | | | | | |
| 0.5 | 9% | 19% | 23% | 31% | 26% | 27% | 30% | 32.1% | 33.0% | 35.7% |
| 1 | 11% | 28% | 25% | 36% | 33% | 36% | 36% | 35.7% | 38.0% | 42.9% |
| 2 | 14% | 26% | 32% | 45% | 35% | 42% | 48% | 46.4% | 49.0% | 53.6% |
| 4 | 27% | 38% | 42% | 61% | 40% | 50% | 57% | 57.1% | 60.0% | 61.9% |
| 30 | 71% | 70% | 62% | 69% | 64% | 60% | 71% | 76.2% | 77.0% | 78.6% |
| Absorbency | | | | | | | | | | |
| Rate Index | | | | | | | | | | |
| 0 to 4 min | 60% | 112% | 123% | 173% | 134% | 155% | 171% | 171.4% | 180.0% | 194.0% |

TABLE 5
Free Swell absorption rate of entire product (zones 1-10)
Free Swell Capacity (g/diaper)

| Product: | | Pampers | Luvs - Ultra | | | | |
|---|---|---|---|---|---|---|---|
| Immersion | Experimental | Baby Dry | Leakguard | Huggies Baby | White Cloud | Experimental | Working |
| time (min) | product A | (10-17 kg) | (10-17 kg) | Shaped (US) | Diaper (10-17 kg) | product B | product T3CM |
| 0.5 | 118 | 382 | 451 | 411 | 343 | 152 | 295 |
| 1 | 142 | 445 | 523 | 426 | 409 | 207 | 401 |
| 2 | 194 | 482 | 615 | 467 | 495 | 287 | 532 |
| 4 | 372 | 510 | 659 | 567 | 553 | 358 | 631 |
| 30 | 681 | 556 | 845 | 768 | 734 | 549 | 773 |

Table 5 provides free swell absorption rate data for the investigated products of Table 1. Table 5 shows that the Working Product T3CM has an free swell which offers comparable performance to currently available products. Even though the Experimental Products A and B have similar free swell capacity as compared to conventional fluff/SAP products, Products A and B failed to provide acceptable performance as indicated in consumer use testing.

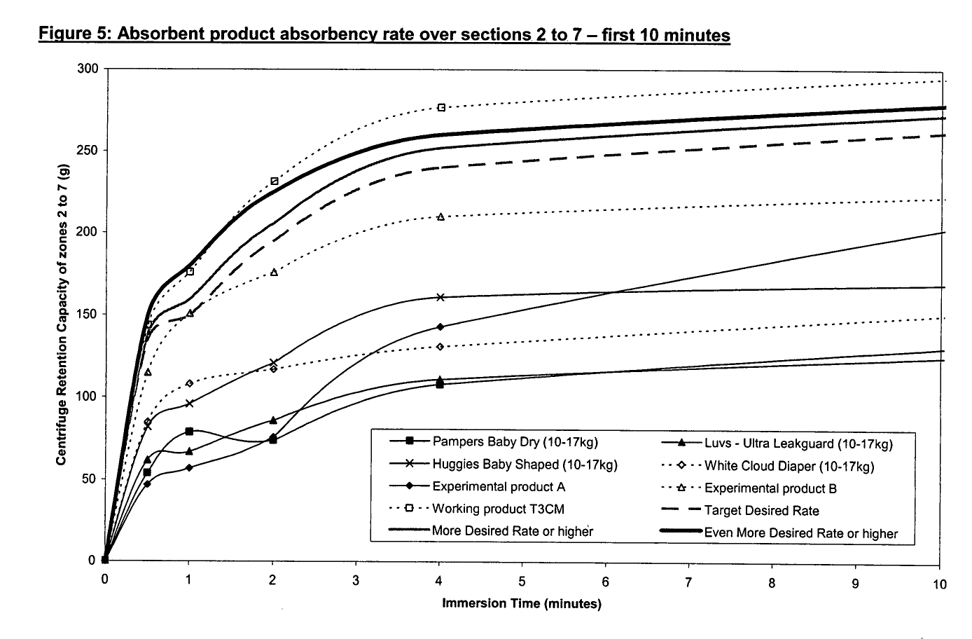

FIG. 5 is a graphical representation of the CRC values over time for the various samples of Table 1. FIG. 4 also illustrates targeted desired rates for embodiments of the present invention.

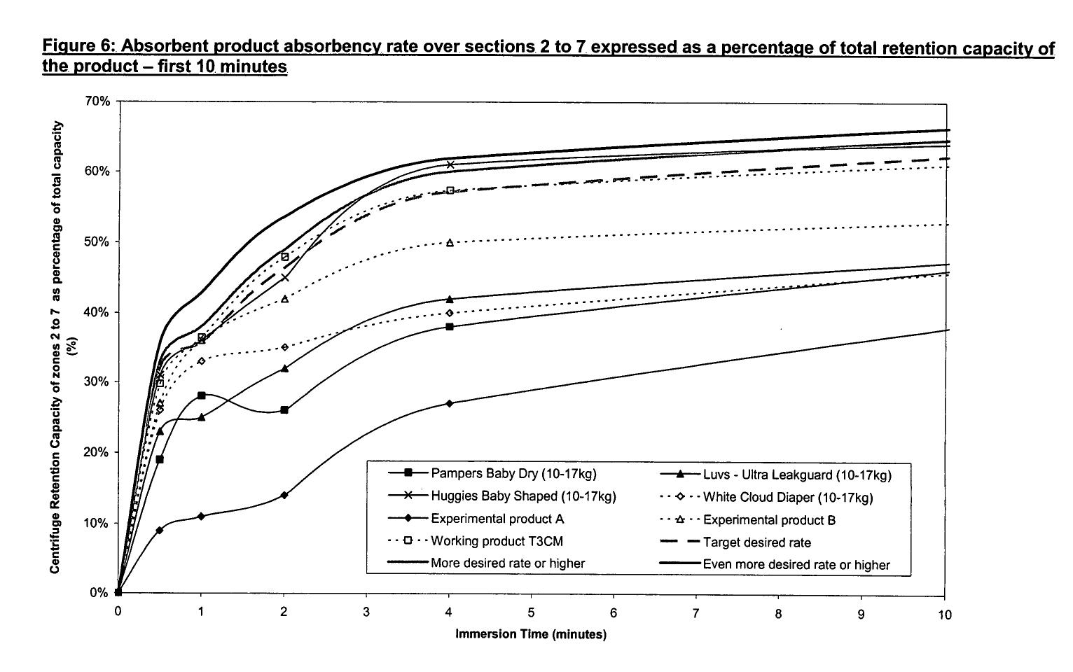

FIG. 6 is a graphical representation of the CRC values in percentage form for the various samples of Table 1. FIG. 5 also illustrates targeted desired rates for embodiments of the present invention.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A disposible absorbent article comprising:
a central absorbent body containing a pulp-less absorbent core, said absorbent core defining an insult target region having liquid absorption characteristics of at least;

| Time | Minimum insult target region liquid |
|---|---|
| (min) | absorption (grams) |
| 0.5 | 135.0 |
| 1 | 150.0 |
| 2 | 195.0 |
| 4 | 240.0 |

2. The disposable absorbent article of claim 1 wherein the insult target region provides liquid absorption characteristics of at least:

| Time | Minimum insult target region liquid |
|---|---|
| (min) | absorption (grams) |
| 0.5 | 138.6 |
| 1 | 159.6 |
| 2 | 205.8 |
| 4 | 252.0 |

3. The disposable absorbent article of claim 1 wherein the insult target region provides liquid absorption characteristics of at least:

| Time | Minimum insult target region liquid |
|---|---|
| (min) | absorption (grams) |
| 0.5 | 150.0 |
| 1 | 180.0 |
| 2 | 225.0 |
| 4 | 260.0 |

4. The disposable absorbent article of claim 1 wherein the absorbent core is defined by zones and wherein the insult target region includes a plurality of the zones.

5. The disposable absorbent article of claim 1 wherein the target insult region has a substantially greater amount of superabsorbent material per unit area as compared to the region outside of the insult target region.

6. The disposable absorbent article of claim 1 wherein the absorbent article is a diaper sized for a 10 kg-17 kg user.

7. The disposable absorbent article of claim 1 wherein the absorbent article is an adult incontinence pad.

8. The disposable absorbent article of claim 1 wherein the insult target region extends across approximately two-thirds of an overall length of the absorbent core.

9. A disposable absorbent article comprising:
a central body containing a pulp-less absorbent core, said absorbent core defining an insult target region extending across the absorbent core, and wherein the insult target region provide liquid absorption characteristics of at least:

| | Percentage absorbed of absorbent |
|---|---|
| Time | core's total liquid capacity |
| (min) | (i.e., 100% is total liquid capacity) |
| 0.5 | 32.1% |
| 1 | 35.7% |
| 2 | 46.4% |
| 4 | 57.1% |

10. The disposable absorbent article of claim 9 wherein the insult target region provides at least following liquid absorption characteristics:

| | Percentage absorbed of absorbent |
|---|---|
| Time | core's total liquid capacity |
| (min) | (i.e., 100% is total liquid capacity) |
| 0.5 | 33.0% |
| 1 | 38.0% |
| 2 | 49.0% |
| 4 | 60.0% |

11. The disposable absorbent article of claim 9 wherein the insult target region provides at least the following liquid absorption characteristics:

| | Percentage absorbed of absorbent |
|---|---|
| Time | core's total liquid capacity |
| (min) | (i.e., 100% is total liquid capacity) |
| 0.5 | 35.7% |
| 1 | 42.9% |
| 2 | 53.6% |
| 4 | 61.9% |

12. The disposable absorbent article of claim 9 wherein the target insult region has a substantially greater amount of superabsorbent material per unit area as compared to the region outside of the insult target region.

13. The disposable absorbent article of claim 9 wherein the absorbent article is a diaper sized for a 10 kg-17 kg user.

14. The disposable absorbent article of claim 9 wherein the absorbent article is an adult incontinence pad.

15. The disposable absorbent article of claim 9 wherein the insult target region extends across approximately two-thirds of an overall length of the absorbent core.

16. A disposable absorbent article comprising:
a central body containing a pulp-less absorbent core and having an insult target region extending across the absorbent core, and wherein the insult target region has an Absorbency Rate Index (ARI) of 700 or greater.

17. The absorbent article of claim 16 wherein the insult target region has an Absorbency Rate Index (ARI) of 750 or greater.

18. The absorbent article of claim 16 wherein the insult target region has an Absorbency Rate Index (ARI) of 815 or greater.

19. A disposable absorbent article comprising:
a central body containing a pulp-less absorbent core and having an insult target region extending across the absorbent core, and wherein the insult target region has a percentage absorbency rate Index (PARI) of 170% or greater.

20. The absorbent article of claim 19 wherein the insult target region has a percentage absorbency rate Index (PARI) of 180% or greater.

21. The absorbent article of claim 19 wherein the insult target region has a percentage absorbency rate Index (PARI) of 190% or greater.

22. The absorbent article of claim 19 wherein the insult target region has an Absorbency Rate Index (ARI) of 700 or greater.

23. A method of manufacturing an absorbent article comprising the steps of:
selecting a backsheet layer having a bodyside surface;
selecting a substantially liquid permeable topsheet layer superposed over the bodyside surface of the backsheet layer;
selecting an absorbent body, said absorbent body providing at least one of the following characteristics:
bent body having an insult target region providing minimum liquid absorption characterstics of:

| Time | Minimum insult target region |
|---|---|
| (min) | liquid absorption (grams) |
| 0.5 | 135.0 |
| 1 | 150.0 |
| 2 | 195.0 |
| 4 | 240.0 |

b) the absorbent core has an insult target region providing at least the following liquid absorption characteristics:

| | Percentage absorbed of |
|---|---|
| | absorbent core's total liquid |
| | capacity |
| Time | (i.e., 100% is total liquid |
| (min) | capacity) |
| 0.5 | 32.1% |
| 1 | 35.7% |
| 2 | 46.4% |
| 4 | 57.1% |

c) the absorbent core has an insult target region having an absorbency rate Index (ARI) of 700 or greater, and
d) the absorbent core has an insult target region having a percentage absorbency rate Index (PARI) of 170% or greater; and
securing the absorbent body between said topsheet layer and said backsheet layer.
